(19) United States
(12) Patent Application Publication
(10) Pub. No.: US 2012/0214187 A1
(43) Pub. Date: Aug. 23, 2012

(54) Method for Enhancing the Sensitivity of Antibody Based Assays

(75) Inventor: Andrew Lees, Silver Spring, MD (US)

(73) Assignee: FFINA BIOLUTIONS, LLC, Silver Spring, MD (US)

(21) Appl. No.: 13/505,232

(22) PCT Filed: Nov. 2, 2010
(86) PCT No.: PCT/US10/55107

§ 371 Date: Apr. 30, 2012

(30) Foreign Application Priority Data
Nov. 2, 2009 (US) 61257282

(51) Int. Cl. G01N 33/566; C07H 21/00; C08B 37/02; C07K 16/00; C07K 14/00; C12N 9/96

(52) U.S. Cl. 435/7.95; 530/350; 435/188; 536/112; 530/391.3; 536/23.1; 436/501

(57) ABSTRACT

There is a constant need for increased sensitivity of antibody-based assays as well as a need for high affinity antibodies. Higher affinity molecules such as antibodies increase the specificity and sensitivity of assays. Both increased sensitivity and higher affinity can be achieved when multiple copies of a binding molecule are coupled to a flexible backbone. According to one embodiment of the invention, multiple copies of a detection molecule, such as, for example, an antibody or antibody fragment, are covalently linked to a high molecular weight water soluble polymer creates multivalent antibody constructs. The affinity or avidity increases markedly as multivalency increases and also, multiple copies of a signal molecule can be similarly added to the polymer. Thus, the construct comprises multiple copies of detection and signal molecules on a high molecular weight, water soluble, flexible polymer. Broadly, the invention comprises a multiplicity of detection molecules (D) and a multiplicity of signal molecules (S) on a polymer (P), such that P_{mw} (Dₙ, Sₘ), where n and m are greater than or equal to 2, and P_{mw}, refers to an average molecular weight of the polymer. These molecules provide for both conventional and nonconventional assays, an increased sensitivity of antigen detection.

### REFERENCE TO RELATED APPLICATIONS

This application claims priority to U.S. Provisional Application No. 61/257,282 filed Nov. 2, 2009, the entirety of which is hereby specifically incorporated by reference.

### BACKGROUND

1. Field of the Invention

This invention is directed to tool and methods of enhancing the sensitivity and avidity of detection assays. In particular, the invention is directed to compositions comprising multiple antibodies and preferably also signaling molecules coupled to a polymer backbone, and to method for the detection of specific antigens with these molecules as well as detection agents such oligonucleotides and streptavidin as an adapter.

2. Description of the Background

Many assays use antibodies as detection reagents due to the exquisite selectivity of antibodies. A very large number of formats for antibody-based assays exist. An ELISA assay is an example of an antibody-based assay system. Methods of performing ELISAs are well known in the art and a variety of formats are currently utilized. Methods of setting up ELISAs are described, for example, in Elisa: Theory and Practice (Methods in Molecular Biology) by John R. Crowther. Humana Press, 1995; Immunoassays: A Practical Approach (Practical Approach Series) James P. Gosling, and Assay Development: Fundamentals and Practice. Ge Wu., John Wiley & Sons, 2010. Other antibody-based assay systems are lateral flow devices. These are described in Lateral Flow Immunoassay, Editors: R. Wong & H. Tse, Humana Press, 2009.

A variety of formats can be used for setting up antibody-based detection systems. For reference, some of these are illustrated in FIGS. 1 and 2. In a typical system, a detection antibody is linked to an enzyme, such as horseradish peroxidase. In another format, the detection antibody is biotinylated. The signal component is a streptavidin-enzyme complex.

In general, an assay system comprises compositions and methods of detection, which provides specificity for the assay, and a means of signaling that detection which provides a readout.

Although ELISA is proved over and over again to be commercially successful, the basic process involves individual components that recognize and bind to each other in a successive process. Increased affinity is dependent on the affinity of each of the individual components as well as the ability of the signal to be read. A need exists for improved affinity and increased signaling which would greatly enhance the usefulness of this already commercially commanding procedure.

### SUMMARY OF THE INVENTION

The present invention overcomes the problems and disadvantages associated with current strategies and designs, and provides new tools and methods for enhancing the sensitivity of detection assays.

One embodiment of the invention is directed to compositions comprising a polymer with an average molecular weight of at least 50 kDa that is both water soluble and flexible to which is coupled to multiple detector or signal molecules at a molar ratio of detector or signal molecules to polymer of at least 10 to 1. Preferably the polymer is high molecular weight form of polyacrylamide, dextran, Ficoll, pullunun, polyethylene glycol, a polyamino acid, or a combination thereof. Preferably, the average molecular weight or the polymer is at least at least 100 kDa, at least 500 kDa, at least 1,000 kDa, at least 2,000 kDa, or at least 5,000 kDa, and the polymer is flexible and bends without breaking at multiple locations along its longitudinal axis. Preferably the multiple detector or signal molecules comprise greater than or equal to five, greater than or equal to ten, greater than or equal to twenty, greater than or equal to thirty, greater than or equal to fifty, greater than or equal to one hundred, greater than or equal to two and fifty hundred, or greater than or equal to five hundred, and the ratio is at least 50:1, at least 100:1, or at least 1,000:1. Preferred sensor or detector molecules include, but are not limited to antibodies or parts thereof, amino acids or peptides, avidin or streptavidin, luminescent molecules, receptor antigens, nucleic acid molecules, fluorescent molecules, radio-labeled molecules, enzyme-linked molecules, or magnetic molecules. Preferably the polymer is coupled to the sensor or detector molecules, or both, by covalent bonds, hydrogen bonds, van der Waals forces, or a combination thereof. Another embodiment of the invention involves a polymer that is water insoluble.

Another embodiment of the invention comprises a polymer with an average molecular weight of at least 50 kDa that is both water soluble and flexible to which is coupled an adaptor such that multiple detector or signal molecules are indirectly coupled to the polymer through the adaptor molecule. Suitable adaptor molecules include, for example, streptavidin, oligonucleotides, oligopeptides, antigens, receptor molecules, and antibodies and parts thereof. Target molecules such as ligands may be coupled to signal molecules such as radio labels, biotin, or enzymes.

Another embodiment of the invention is directed to methods for the detection of an antigen comprising: contacting a sample suspected of containing the antigen with a composition comprising a polymer with a molecular weight of at least 50 kDa that is both water soluble and flexible to which is coupled to multiple detector or signal molecules at a ratio detector or signal molecules to polymer of at least 10 to 1; incubating the sample and the composition for a period of time to form antigen-detector or signal molecule complexes; detecting the presence of complexes formed after incubation; and determining whether the antigen is present in the sample from detecting the presence of the complexes. Preferably the sample is a biological sample and the antigen is selected from biological molecules, peptides, proteins, receptors, or indicator molecules. Also preferably, detection is quantitative, although qualitative detection is preferred. Preferably contacting comprises mixing the sample with the polymer, and contacting may comprise mixing equal parts by weight of sample with polymer, incubation is for at least 5 seconds at room temperature or above, or for at least 5 minutes at above room temperature. Preferably, the unbound polymer is removed prior to detecting the presence of complexes. Also preferably, avidity of detection is increased at least two fold as compared to detection in an ELISA, and more preferably at least ten fold as compared to detection in an ELISA.

Other embodiments and advantages of the invention are set forth in part in the description, which follows, and in part, may be obvious from this description, or may be learned from the practice of the invention.

### DESCRIPTION OF THE FIGURES

FIG. 1. Prior art ELISA formats: (a) direct assay; (b) indirect assay; (c) sandwich assay; and (d) competitive assay.

FIG. 2. Prior art lateral flow device formats: (a) lateral flow immunoassay test strip; (b) direct solid-phase immunoassay; and (c) competitive solid-phase immunoassay.

FIG. 3. Prior art assay format using oligonucleotides as the detector component.

FIG. 4. Streptavidin-(HRP/polymer).

FIG. 5. Assembly of a detection/signaling polymer using a streptavidin polymer.

FIG. 6. Streptavidin/polymer adapter.

FIG. 7. Synthesis of antibody polymer construct with signal enzyme.

FIG. 8. Synthesis of antibody polymer construct with signal molecule.

FIG. 9. Binding of enhanced multivalency of the antibody and by flexibility of the polymer.

### DESCRIPTION OF THE INVENTION

There is a constant need for increased sensitivity of antibody-based assays. A further issue in antibody-based assays is the need for high affinity antibodies. In general, higher affinity antibodies increase the specificity and sensitivity of assays. Previous approaches include making aggregates of the antibody. For example, the antibody is cross-linked with itself or to particles. However, these aggregates are not flexible which limits their effective multivalency. Another mode puts streptavidin on a low molecular weight polymer and (Polyacrylamide-streptavidin: a novel reagent for simplified construction of soluble multivalent macromolecular conjugates, J Immunol Methods 1989, Jun. 21; 120(2):233-239.). In contrast, the invention uses high molecular weight polymers to achieve very high multiplicity of the signaling component. Multiples of at least two-fold, five-fold, ten-fold, twenty-fold, one hundred-fold and higher are preferred.

It has been surprisingly discovered that both increased sensitivity and higher affinity can be achieved when multiple copies of a binding molecule are coupled to a flexible backbone. According to the invention, multiple copies of a detection molecule (e.g., detector or sensor molecule or other chemical agents), such as, for example, an antibody or antibody fragment, are covalently linked to a high molecular weight water soluble polymer (e.g., natural or synthetic) creates multivalent antibody constructs. The affinity and/or avidity increases markedly as multivalency increases and also, multiple copies of a signal molecule can be similarly added to the polymer. Thus, the construct comprises multiple copies of detection and signal molecules linked directly or indirectly to a high molecular weight, water soluble, flexible polymer. More broadly, the invention comprises a multiplicity of detection molecules (D) and a multiplicity of signal molecules (S) on a polymer (P), such that P_{mw} (Dₙ, Sₘ), where “n” and “m” are greater than or equal to 2 and P_{mw} refers to an average molecular weight of the polymer. The compositions and methods of the invention utilize high molecular weight polymers to achieve very high multiplicity of the signaling component. Multiples of at least two-fold, five-fold, ten-fold, twenty-fold, one hundred-fold and higher are preferred.

One embodiment of the invention is directed to compositions comprising multiple copies of detection and signal molecules both coupled directly or indirectly to a high molecular weight, water soluble, flexible polymer. Adapater molecules, such as for example streptavidin or biotin, facilitate linking of the antibodies, signal molecules and other chemicals to the polymer, can also be coupled to such polymers.

Another embodiment of the invention is directed to methods for the detection of an antigen comprising contacting a sample with a composition of the invention. These methods provide for increased avidity of the detection signal, such as an antibody, and significantly increases the response signal. As the polymer is preferably flexible, the arms of these multiple detection molecules can easily reach and bind their target ligands, thereby enhancing the apparent affinity of the detection molecule for its target. Further, constructs can be constructed in situ and without the need for multi-step procedures, thus, simplifying and greatly reducing the expense and complications necessary with conventional multi-step processes. The construct of the invention increases the signal response in antibody based assays and increases the effective affinity (avidity) of antibodies. The invention allows for these two parameters to be independently varied.

In another embodiment of the invention, constructs comprise streptavidin bound to a high molecular weight polymer and then adding biotinylated detection and biotinylated signal molecules that bind to sites of the streptavidin molecule. This allows for the incorporation of very high molecular weight polymer and, thus, the incorporation of large numbers of detection and signaling molecules.

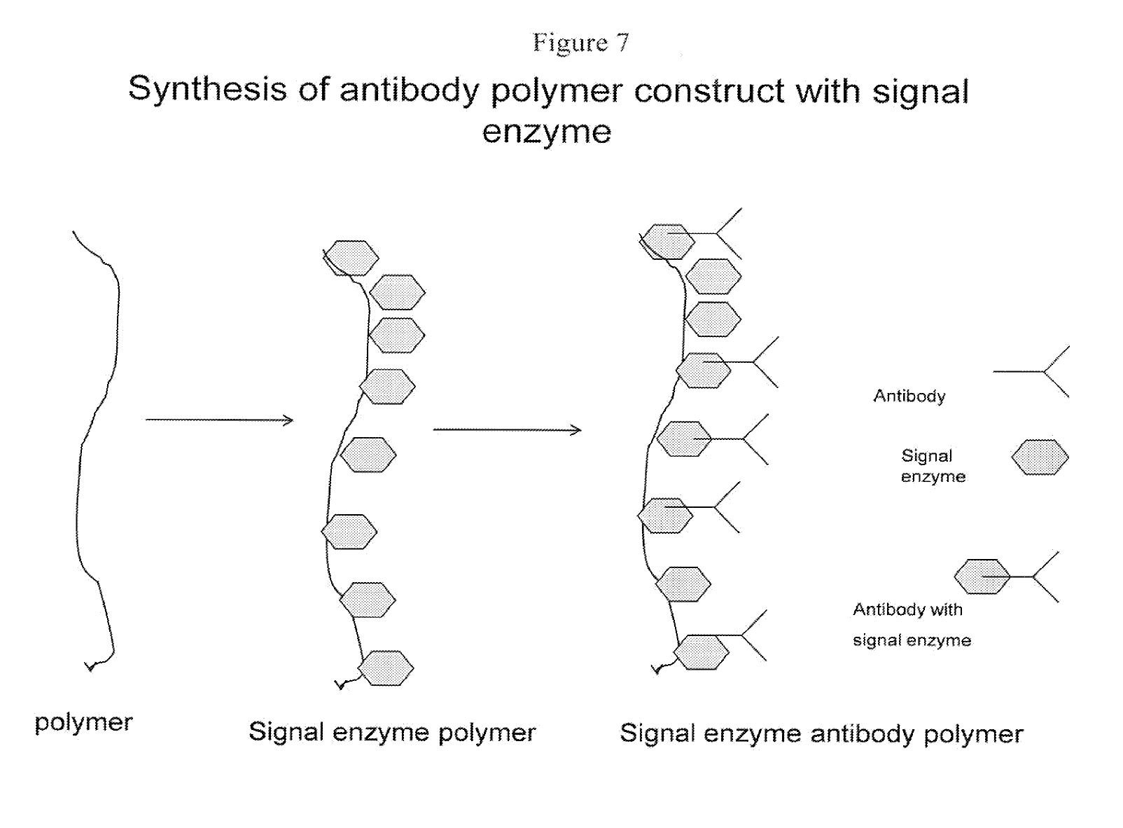

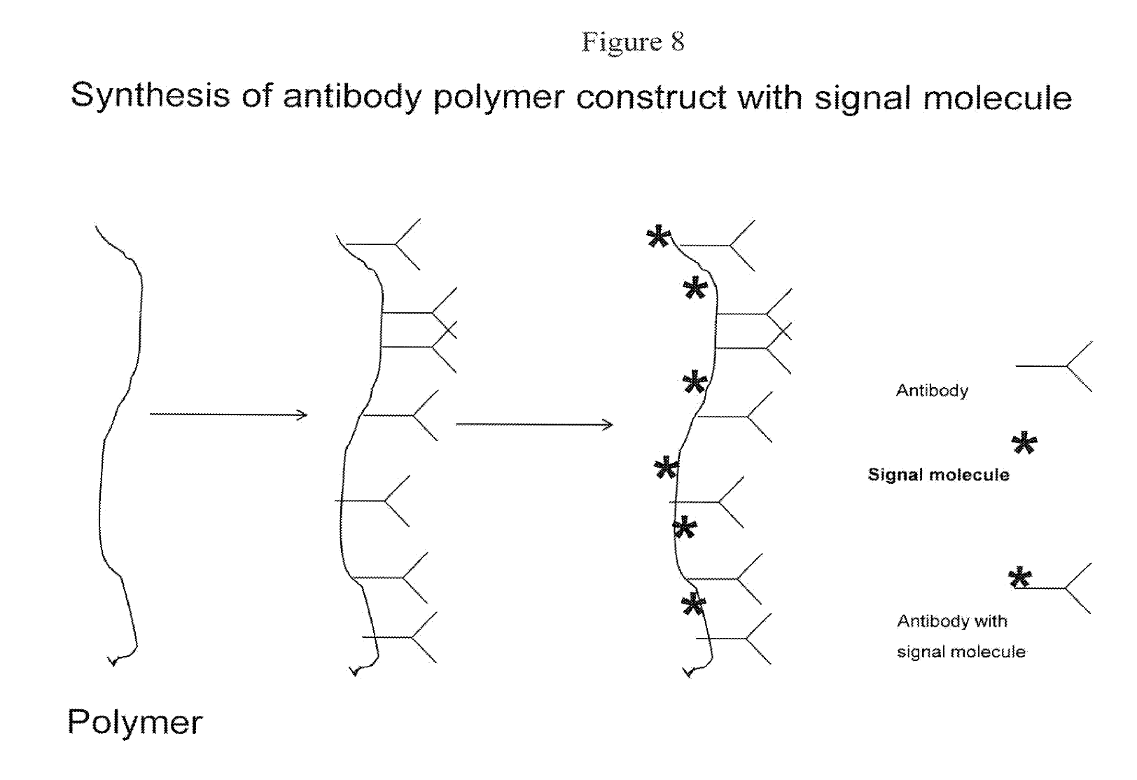

One method of detection comprises an antibody to which a signal-generating enzyme has been covalently linked. Another method is to covalently link a signal molecule, for example a fluorescent substance, to the antibody. In another example, the signal-generating enzyme or signal molecule is attached to an antibody specific for the analyte antibody. In other embodiments an adaptor molecule, such as for example biotin or avidin, coupled to the polymer or alternatively the signal or detection molecules, facilitates detection of the analyte antibody. Preferably, signal detection molecules are added to the antibody-polymer construct (e.g., see FIGS. 7 and 8). For example, the construct can be biotinylated or fluorescently labeled. A signal-generating enzyme can also be added. The detection molecules can be added before or after linking the antibody to the polymer and can be linked to the antibody, to the polymer or to both. Preferably, the signal molecule is linked to the polymer first and the detection molecule added second. Preferably, the signal molecule or the detection molecule is first covalently linked to the polymer and the other is linked to it. Thus, the second component may be linked indirectly to the polymer. The signal molecule and the detection molecule can be linked to each other before linking to the polymer.

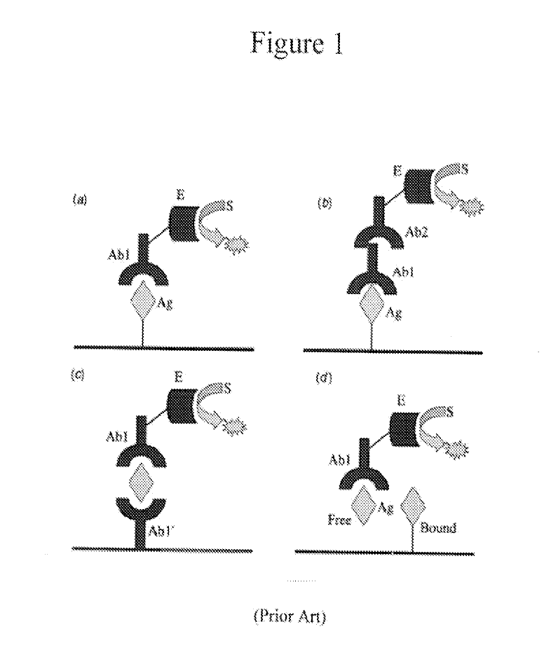

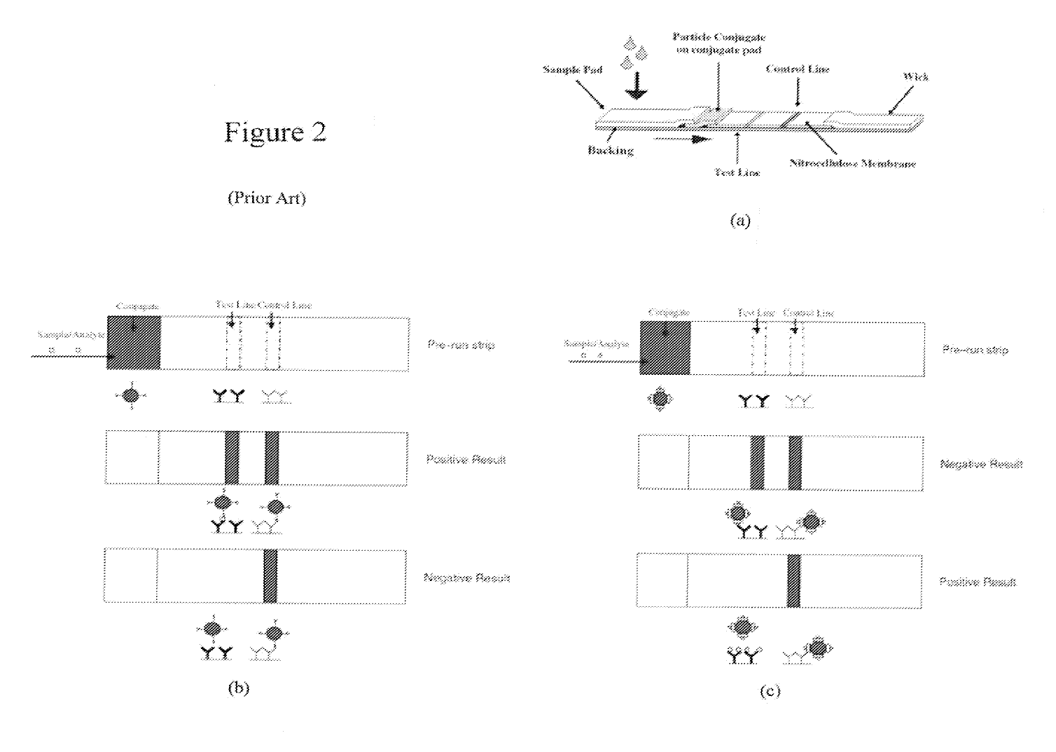

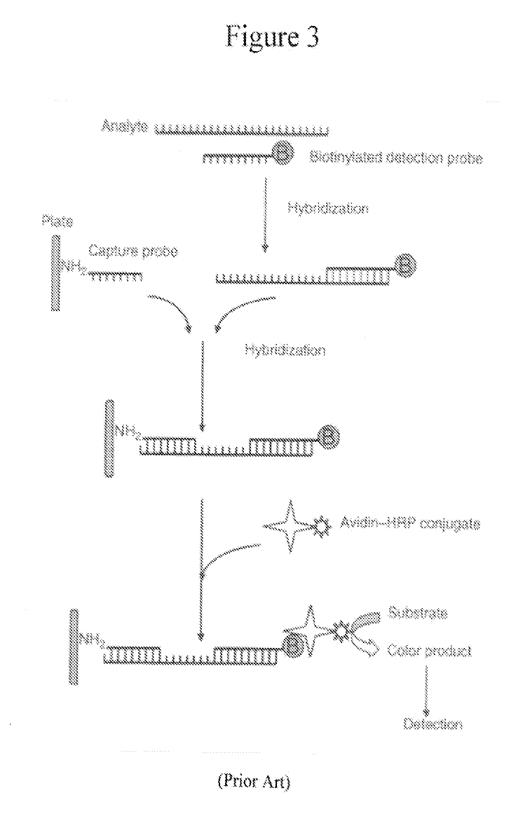

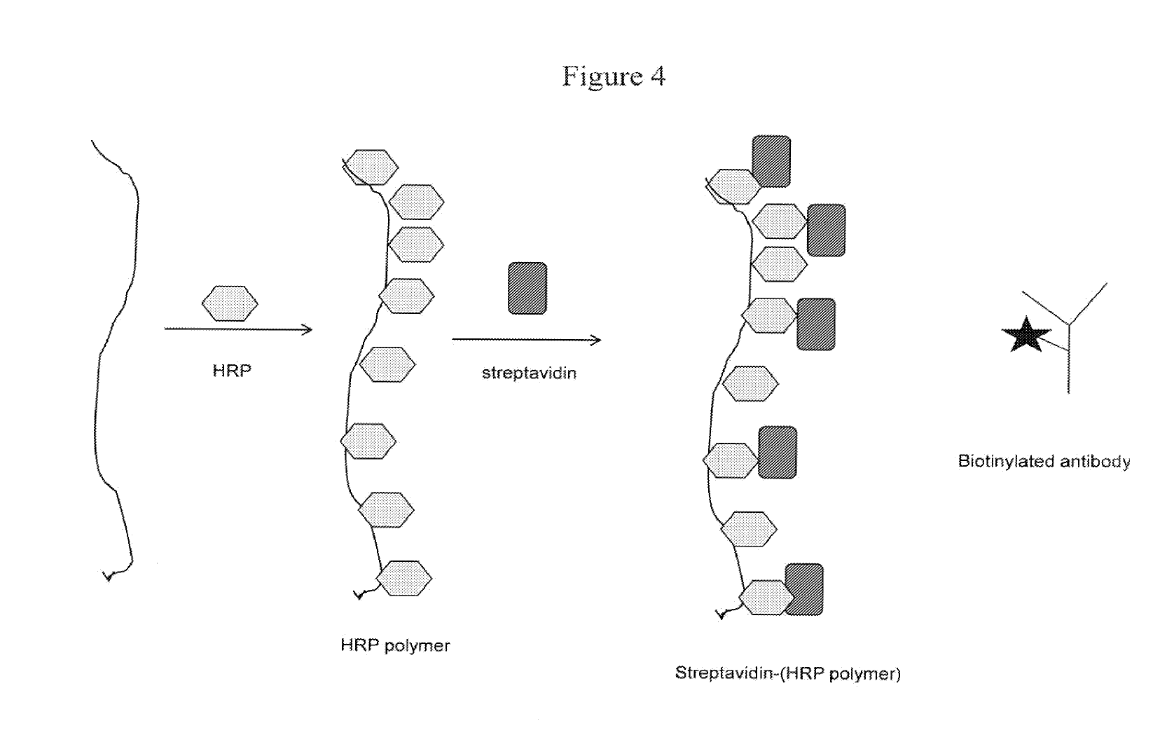

An example of a typical ELISA is shown in FIG. 1 and the use of the invention in an ELISA system is shown in FIG. 4. An example of a typical lateral flow assay is shown in FIG. 2. The use of Watson-Crick oligonucleotide pairing in an assay system is shown in FIG. 3. In each case, there is a detection component, which provides specificity and a signaling component, which gives a readout indicating the detection. The invention, which comprises multiple copies of detection and signaling components on a polymer, substitutes for the detection/signal system indicated in these examples.

The molecular weight of the polymer should be such as to permit multiple copies of the antibody to be bound but is otherwise not limited. For example, ≧25 kDa, ≧50 kDa, ≧100 kDa, ≧500 kDa. Preferably, the polymer is ≧1000 kDa, ≧2000 kDa, ≧5000 kDa or more.

Preferably, detection component not linked to the polymer, such as unlinked antibody, is removed from the polymer construct, for example, by size exclusion chromatography or tangential flow filtration.

The polymer is preferably size fractionated to reduce its polydispersity, for example, removing low and/or high molecular weight polymers. Examples of polymers include, for example, polyacrylamide, dextran, Ficoll, pullunun, polyethylene glycol, polyaminoacids and compounds that are constructions and combinations thereof.

A variety of chemical methods can be used to link, preferably covalently, the signal and detection components to the polymer. Methods are described, for example, in Lees et al, Enhanced immunogenicity of protein-dextran conjugates. I. Rapid stimulation of large specific antibody responses to poorly immunogenic molecules. Vaccine 12:1160, 1994; Mond, J. J. and A. Lees. Dual immunogenic construct. U.S. Pat. No. 5,585,100, issued Dec. 17, 1996: U.S. Pat. No. 5,955,079, issued Sep. 21, 1999; Lees, A., Producing immunogenic constructs using soluble carbohydrates activated via organic cyanylating reagents. U.S. Pat. No. 5,651,971, issued Jul. 29, 1997; U.S. Pat. No. 5,693,326, issued Dec. 2, 1997; U.S. Pat. No. 5,849,301, issued Dec. 15, 1998; Lees, A. Use of amino-oxy functional groups in the preparation of protein-polysaccharide conjugate vaccines. U.S. Patent Application Publication No. 2005/0169941, filed Jan. 27, 2005. Other methods include such as, for example, the hydrazone reagents sold by Solulink, 9853 Pacific Heights Blvd. Suite H, San Diego, Calif. 92121. Conjugation methods are also described in Bioconjugate Techniques, G T Hermanson, Academic Press 2008 and Bioconjugation Protocols: Strategies and Methods (Methods in Molecular Biology) Christof M. Niemeyer (Editor).

Preferably, the polymer is modified or functionalized before adding the signal and detection molecules. For example dextran may be functionalized with amines using the method of Inman, J K, J Immunol. 1975 February;114(2 Pt 1):704-9. Preferably, before being linked to the polymer, the antibody is modified with signal detection molecules and/or functional groups to facilitate conjugation. Similarly, a variety of means can be used to add the signal molecules to the polymer or the antibody. Examples of chemistries uses to modify antibodies can be found in GT. Hermanson,

Bioconjugate Techniques, Academic Press, 2008. CDAP chemistry (Lees, A., Producing immunogenic constructs using soluble carbohydrates activated via organic cyanylating reagents. U.S. Pat. No. 5,651,971, issued Jul. 29, 1997; U.S. Pat. No. 5,693,326, issued Dec. 2, 1997; U.S. Pat. No. 5,849,301, issued Dec. 15, 1998) is particularly useful for the preparation of polymer conjugates. Examples of signal enzymes include, but are not limited to, catalase, horse radish peroxidase, alkaline phosphatase, glucose oxidase, and combinations thereof. Examples of signal molecules include, among others, electrochemiluminescent reagents (available from MesoScale), fluorescent reagents such as Alexafluor and Cytofluor dyes and fluorescent proteins such as green fluorescent protein and phycoerythrin. Alternatively, the signal molecule may be radiolabel. The number of antibody molecules per polymer is at least two and preferably three, five, seven, ten, twenty, fifty, one hundred or more. The number of signal molecules per polymer is at least two and preferably three, five, seven, ten, twenty, fifty, one hundred or more. The method is applicable to multiplexed antibody based assays when different signal molecules are used in association with different detection antibodies. Streptavidin can also be used as an adapter the detection component.

Preferably, the method is a universal detection system for a class of analytes. For example, an anti-mouse IgG antibody is linked to the polymer along with multiple copies of the signal enzyme, creating a detection reagent with higher affinity and with an amplified signal. The anti-mouse IgG antibody acts as an adaptor, allowing any mouse antibody to be detected.

Other detection reagents besides antibodies can be used in additional embodiments of the invention. For example, multiple copies of oligonucleotides can be attached to the polymer and through Watson-Crick pairing (hydrogen bonding), bind to complementary nucleic acids. An example of a hybridization based detection system is shown in FIG. 3 and discussed in Ligand-binding assays, Khan and Findlay (ed), Chapter 13, Wiley, 2010. Aptamers (e.g., oligonucleic acid or peptide molecules that bind to a specific target molecule) can also be attached to the polymer for use as the detection component. Use of aptamers is described, for example, in Nucleic Acid and Peptide Aptamers: Methods and Protocols (Methods in Molecular Biology), Gunter Mayer (ed), Humana Press, 2009. Oligonucleotides can also be used as a component of the signaling moiety. Oligonucletotides on the polymer can be amplified using PCR methods.

In another preferred embodiment of the invention, an adaptor molecule is used to make the polymer in situ. Example: A streptavidin polymer is made, along with biotinylated signal enzyme and biotinylated detection antibody. Combining these species in different ratios produces a final polymer of signaling enzyme and detection antibody in different ratios.

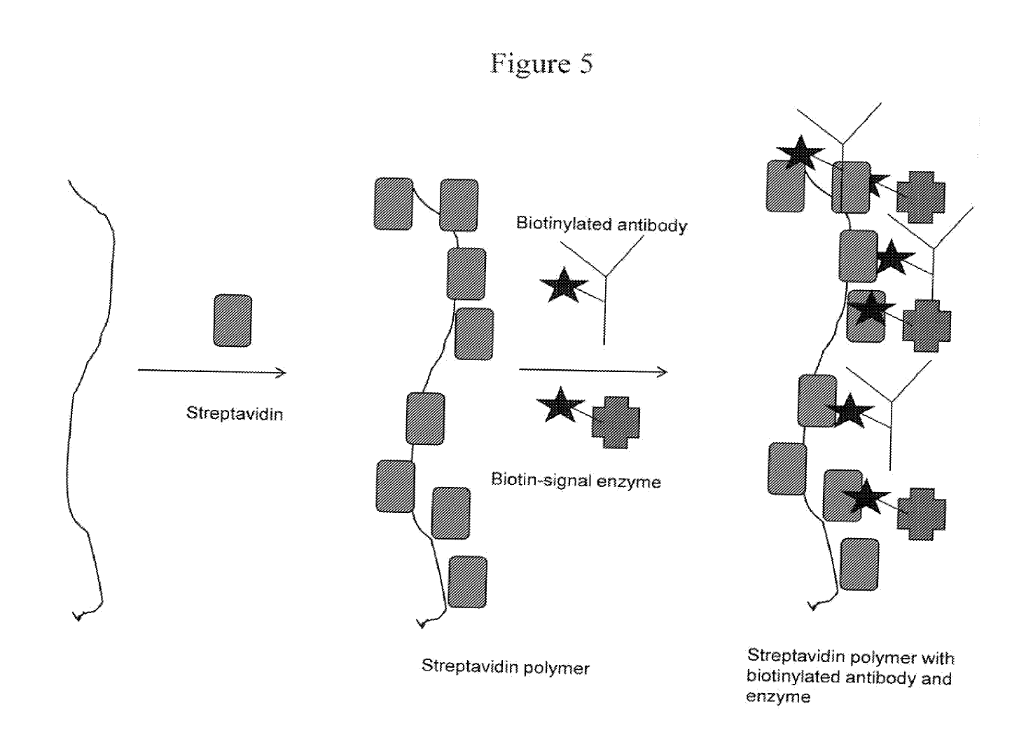

In another preferred embodiment, only one of the two biotinylated components is added, leaving additional biotin binding sites available. For example, biotinylated antibody is added to the streptavidin polymer. Free antibody is removed, leaving an antibody-polymer which is used in a detection assay. In a second step, biotinylated signal enzyme is added which binds to free biotin binding sites on the streptavidin. (FIG. 5).

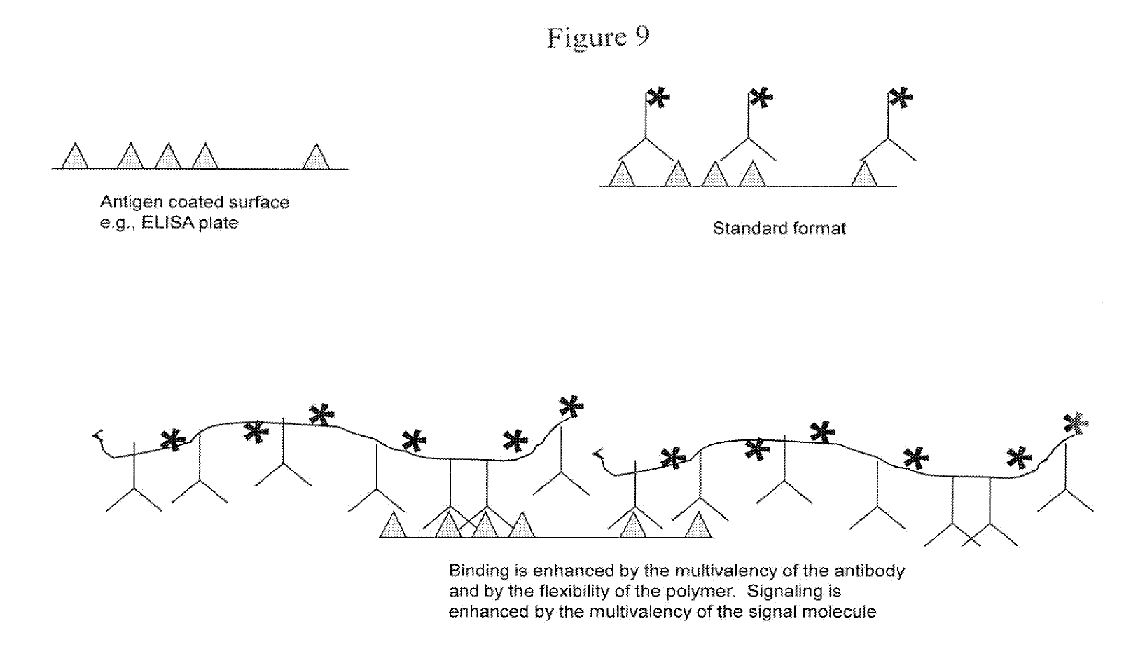

In another preferred embodiment, the biotinylated detection antibody is bound to its target. The steptavidin polymer is then added, followed by the addition of the signal enzyme. This is illustrated in FIGS. 5, 6 and 7. The use of the streptavidin polymer allows one to take advantage of the polymeric enhancement features of the invention without the need to individually synthesize a signal/detection polymer for each detector molecule. As illustrated in FIGS. 8 and 9, the method of the invention is not limited to avidin/biotin coupling. The methods and compositions of the invention may include any polymer with multiple sites for binding of agents that provide for signaling and detection.

The following examples illustrate embodiments of the invention, but should not be viewed as limiting the scope of the invention.

### EXAMPLES

Dextran polymers are available from Sigma Aldrich. Dextran is assayed using the resorcinol sulfuric acid assay of Monsigny et al. (Anal Biochem. 1988 December;175(2):525-30). Protein is assayed from its absorbance and extinction coefficient or using the MicroBCA assay (Pierce). Catalase and HRP concentrations are determined from their absorbance at 405 nm using extinction coefficients of 1.51 and 2.27 AU/mg/ml, respectively. An extinction coefficient of 3.2 AU/mg/ml was used to determine the concentration of streptavidin.

### Example 1

T2000 dextran (Sigma) is size fractionated on an S400HR gel filtration column (GE Healthcare) as described by Lees et al. (Enhanced immunogenicity of protein-dextran conjugates. I. Rapid stimulation of large specific antibody responses to poorly immunogenic molecules. Vaccine 12:1160, 1994) to prepare high molecular weight dextran (HMWdex). HMWdex is then functionalized with amino groups as described by Inman (Thymus-independent antigens: the preparation of covalent, hapten-ficoll conjugates. Inman J K. J Immunol. 1975 February;114(2 Pt 1):704-9) and the detector antibody linked as described by Lees et al. Vaccine 12:1160, 1994. The antibody dextran construct is then biotinylated using NHS biotin. A streptavidin-HRP complex is used as the signaling component.

### Example 2

HMWdex is prepared at 10 mg/ml in water and activated with CDAP. After 30 seconds, the pH is raised to ca 9 with triethylamine and at 2.5 min, catalase is added at a ratio of 1 mg catalase per mg dextran and the pH maintained at pH 9. After an overnight reaction, the solution is concentrated using an Amicon Ultra 15 device with a 30 kDa cutoff and the unconjugated catalase removed by gel filtration on a Superdex 200 column to yield a CATdex conjugate. The conjugate is then concentrated to about 10 mg/ml.

The catalase-dextran conjugate is then functionalized with bromoacetyl groups using NHS bromoacetate. The antibody is thiolated using SPDP and deprotected with DTT. The bromoacetylated-CATdex and the thiolated antibody are combined at pH 9 in the presence of 5 mM EDTA and reacted overnight. The unconjugated antibody is removed by gel filtration.

### Example 3

Same as example 1 except that the antibody dextran conjugate is labeled with Alexa 488 fluorescent dye (Invitrogen).

### Example 4

Same as example 1 except the antibody dextran conjugate is labeled with a lanthanide europium reagent such as TCI America product #A2083 ATBTA-Eu3+.

### Example 5

Very high molecular weight dextran (VHMWdex) is prepared using 5-40,000 kDa crude dextran (Sigma Aldrich product #D5501). The material is solublized in water at 5 mg/ml and centrifuged to clarify. The supernatant is then filtered through a coarse filter and then a 0.2 micron filter. Antibody-VHWMWdex polymer is then made as in examples 1 and 2.

### Example 6

Anti-mouse or anti-human IgG antibody is covalently linked to dextran as in examples 1 and 2.

### Example 7

Anti-mouse, anti-human and anti-rabbit IgG antibodies are all covalently linked to dextran as in examples 1 and 2.

### Example 8

Recombinant phycoerythrin is linked to the polymer. An antibody is subsequently linked.

### Example 9

Horse radish peroxidase is covalently linked to an antibody. The HRP-antibody couples are then linked to the polymer.

### Example 10

Streptavidin is covalently linked to the polymer.
Dextran polymers are available from Sigma Aldrich. Dextran is assayed using the resorcinol sulfuric acid assay of Monsigny et al. (Anal Biochem. 1988 December;175(2):525-30). Protein is assayed from its absorbance and extinction coefficient or using the MicroBCA assay (Pierce). Catalase and HRP concentrations are determined from their absorbance at 405 nm using extinction coefficients of 1.51 and 2.27 AU/mg/ml, respectively. An extinction coefficient of 3.2 AU/mg/ml was used to determine the concentration of streptavidin. For antibodies, an extinction coefficient of 1.4 AU/mg/ml was used.

### Example 11

### Preparation of Streptavidin-(HRP-Very High Molecular Weight Dextran) Conjugate.

Preparation of amino-HRP. HRP contains only a few free amino groups. To have a sufficient number of free amines for coupling to the polymer and for linking the detection component, aminated-HRP (or amino-HRP) was prepared as generally described (U.S. Pat. No. 5,039,607, p 14). In brief, 359 mg HRP (BBI Enzymes #HRP-4) was solubilized in 20 ml of 0.1 M pyridine-HCl, pH 5. 1.5 g of ethylenediamine 2HCl was added and the solution adjusted to pH 5 with 0.1 M NOH. 150 mg of EDC (Sigma product #E6383) was solublized in 1 ml water and added to the solution. The solution was stirred for 2 hrs at room temperature, maintaining the pH at 5. The solution was then extensively dialyzed against 5 mM sodium borate, 150 mM NaCl, pH 9 (Borate buffer) to remove excess reagents. This product is termed amino-HRP.

Preparation of VHMWdextran. Dextran (5-40,000 kDa industrial grade dextran, Sigma D5501) was microfluidized at 5000 psi (Microfluidizer Corp), centrifuged and filtered. The peak molecular weight was estimated as 10,000 kDa by dynamic light scattering using dextran standards. This product was termed very high molecular weight dextran (VHMWdextran).

Conjugation of amino-HRP to VHMWdextran. 16 mg of VHMWdextran was prepared at 8 mg/ml in water. At time zero, 16 mg of CDAP (Research Organics) was added from a 100 mg/ml stock in acetonitrile. At 30 sec the pH was raised to 9 with 0.25 M NaOH. At 3 min, 20 mg of amino-HRP in 1 ml of borate buffer was added. After 2 hrs, the conjugate was dialyzed against saline.

The HRP concentration, determined from the absorbance at 405 nm, was 3.7 mg/ml. Dextran, determined using the resorcinol/sulfuric acid assay, was 5.4 mg/ml.

The ratio of absorbance at 280:405 nm was determined as 1.02. This product is called HRP/VHMWdextran.

The enzyme activity of the HRP, measured by the consumption of H₂O₂ at 240 nm, was minimally affected by the conjugation process. The figure compares the activity of two HRP-dextran conjugates with that of the starting HRP and indicates H₂O₂ consumption of the conjugates was similar to that of the starting HRP.

Conjugation of streptavidin to HRP/VHMWdextran. 100 ul of 1 M HEPES was added to 1.2 ml of the HRP/VHMWdextran solution and the pH adjusted to 7.5. 25 ul of 0.1 M GMBS (Molecular BioSciences #98799) in NMP was added. After about 1 hr, the pH was reduced by the addition of 100 ul 1 M sodium acetate, pH 5, plus 25 ul 1 M HCl and then dialyzed overnight against 2 liter of 10 mM sodium acetate, 150 mM NaCl, pH 5. 8.5 mg of streptavidin (Prozyme, #SA10) was solubilized in 850 ul of 1 M HEPES, pH 8 and 23 ul of 0.1 M SPDP (Molecular BioSciences, Inc. #67432) added. After one hr, 100 ul of 1 M sodium acetate, pH 5 was added and the pH adjusted to 6.5 with HCl. The solution was then made 25 mM dithiothreitol. After a 30 min incubation, the solution was desalted on a G25 column equilibrated with 10 mM sodium phosphate +5 mM EDTA, pH 6.8 and the void volume fractions concentrated using an Amicon Ultra 4 10 kDa cutoff device.

The labeled HRP/VHMW-dextran and streptavidin were combined. After approximately 2 hrs, the reaction was quenched by making the solution 10 mM iodoacetamide, pH 9. After 1 hr incubation, unconjugated streptavidin was removed by gel filtration on an S300HR column (GE Healthcare) equilibrated with PBS.

The dextran concentration was determined using the resorcinol/sulfuric acid assay. The absorbance at 405 nm was used to calculate the HRP concentration. The HRP/VHMWdextran 280/405 nm ratio was used to determine the contribution of the HRP/VHMWdextran at 280 nm and the streptavidin concentration calculated from the remainder, using the extinction coefficient for streptavidin (1 mg/ml per 3.2 AU at 280 nm). The conjugate was determined to have approximately 150 HRP per 10,000 kDa polymer of dextran and 0.7 mole streptavidin per mole HRP. This product is streptavidin-(HRP/VHMWdextran).

### Example 12

Biotin-(HRPIVHM-dextran). VHMWdextran was also prepared by a second method. Dextran (5-40,000 kDa industrial grade dextran, Sigma D5501) was solubilized at 10 mg/ml, centrifuged and filtered through a 0.45 u device. Amino-HRP, prepared as described above, was linked to the dextran using CDAP chemistry (U.S. Pat. No. 5,651,971). The conjugate was purified by size exclusion chromatography and concentrated using an Amicon Ultra15 device with a 10 kDa cutoff. HRP concentration was determined from the absorbance at 405 nm and the dextran concentration determined using the resorcinol/sulfuric acid assay. The purified conjugate contained 0.38 mg HRP/mg dextran. The conjugate was biotinylated as follows. 13.8 ul of 0.1 M sulfo-NHS-LC biotin (Pierce #21335) was added to a solution of 2.5 ml of the conjugate (0.5 mg/ml HRP) +0.1 ml 1 M HEPES pH 8, reacted overnight and then dialyzed into PBS to remove the free biotin.

The product was biotin-(HRP/VHMWdextran).

### Example 13

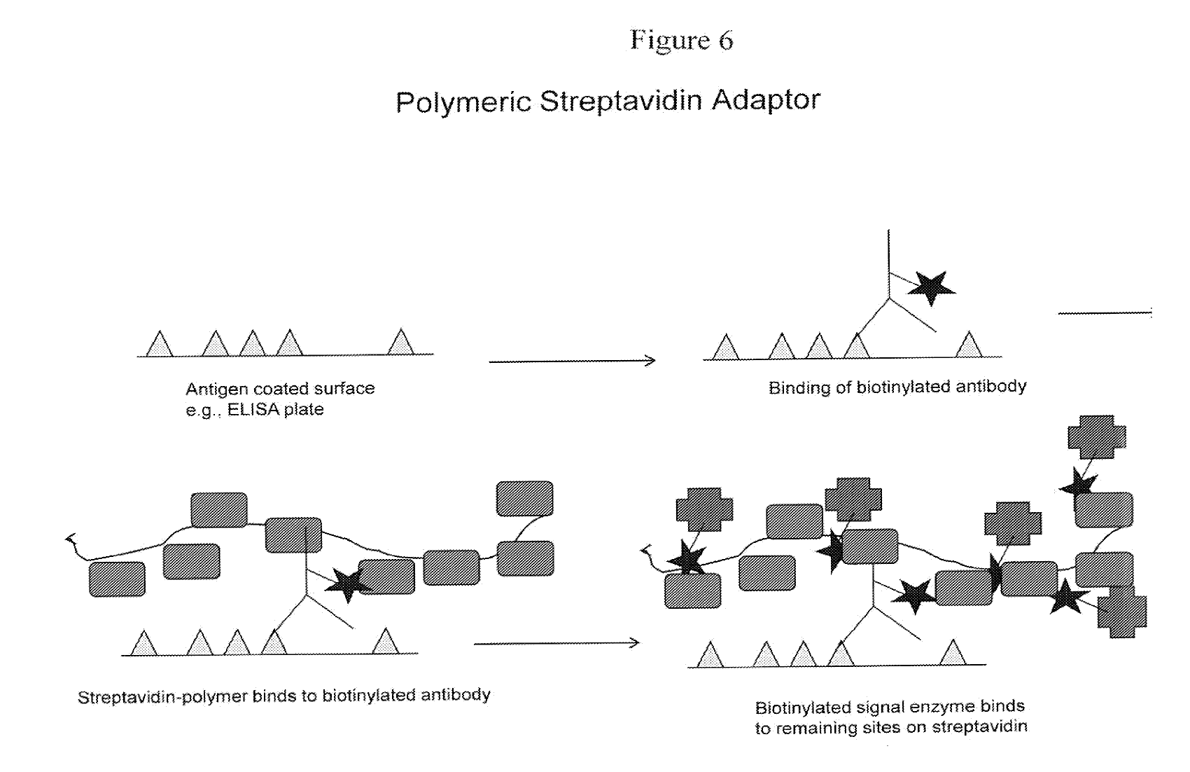

Antibody-(HRP/HMWdextran). High molecular weight dextran (HMWdextran) was prepared from 2000 kDa dextran (Sigma #95771 or T2000 dextran GE Healthcare, no longer available) by fractionation on an S400HR gel filtration column (GE Healthcare) to yield polymer with an average molecular weight of about 2000 kDa (e.g., see FIG. 6). Amino-HRP was prepared as above and linked to the HMWdextran using CDAP as generally described above. The HRP-HMWdextran was labeled with GMBS as described above.

A 1 ml solution of a monoclonal antibody is prepared at 10 mg/ml in 0.1 M HEPES, pH 8 and 13.3 ul of SPDP is added (0.1 M in NMP). After one hr, 100 ul of 1 M sodium acetate, pH 5 is added and the pH adjusted to 6.8. After 30 min of incubation, the solution is desalted on a G25 Sephadex column (GE Healthcare), equilibrated with 10 mM sodium phosphate, 5 mM EDTA, 150 mM NaCl, pH 6.8. The void volume fraction is concentrated to 10 mg/ml using an Amicon Ultra 4 30 kDa cutoff device.

The labeled HRP/HMWdextran and antibody are combined. After a two hr reaction, the solution is made 10 mM in iodoacetamide and the pH raised to 9. Free antibody is removed on an S400HR size exclusion column (GE Healthcare). This product is Mab-(HRP/HMWdextran).

### Example 14

Antibody-(Catalase-VHMW-dextran). Bovine catalase (Worthington Biochemical #LS001898) was desalted into 5 mM borate, 150 mM NaCl +0.1% polysorbate 80, pH 10 and concentrated using an Amicon Ultra 15 30 kDa cutoff device. VHMW dextran (8 ml), prepared by the second method was made 6 mg/ml in a borate buffer and adjusted to pH 9.7 with triethylamine (TEA). At time zero, 489 ul of CDAP (100 mg/ml in acetonitrile) was added and 30 seconds later, 15 ul of TEA added. At 2.5 min, 3 ml of bovine catalase (16 mg/ml in 5 mM sodium borate, 150 mM NaCl, pH 10) was added. The final pH was 9.5. The reaction was allowed to proceed overnight. It was then made 0.1% polysorbate 80, concentrated with an Amicon Ultra 15 30 kDa cutoff device and fractionated on an S400HR size exclusion column (GE Healthcare).

The absorbance at 405 and 280 nm was determined The catalase concentration was determined from its extinction coef (1 mg/ml =1.51 AU at 405 nm). The dextran concentration was determined with the resorcinol/sulfuric acid assay. The catalase concentration was 5.6 mg/ml and 1.3 mg catalase/mg dextran.

0.5 ml of 1 M HEPES, pH 8 was added to 1.7 ml of the catalase-dextran solution was readjusted to pH 8. 20 ul of 0.1 M succinimidyl bromoacetate (Molecular BioSciences, #22080) added and solution incubated for 2 hr and then desalted using an Amicon Ultra15 30 kDa cutoff device using 10 mM sodium borate +150 mM NaCl, pH 9 and concentrated to a volume of 1.6 ml. A 10 mg/ml solution of a monoclonal antibody was prepared in 0.5 M HEPES, pH 8 and 15 ul of SPDP (0.1 M in NMP) was added. After 1.5 hr, 200 ul of 1 M sodium acetate was added, the pH adjusted to 6.4 and the solution made 25 mM in DTT. After 30 mM, the solution was desalted on a G25 column (GE Healthcare) equilibrated with PBS +5 mM EDTA, pH 6.8. The void volume fractions were concentrated to ˜0.5 ml using an Amicon Ultra 15 30 kDa device. This yielded a thiolated antibody preparation of 15.9 mg/ml.

100 ul of 0.1 M sodium borate pH 9 was added to the thiolated antibody and the pH adjusted to 9.3 and the bromoacetylated catalase/VHMWdextran added. The mixture was allowed to react overnight at 4° C. The reaction mixture was centrifuged to remove particulate matter and then quenched by the addition of 50 ul of 50 mM mercaptoethanol followed by 50 ul of 0.5 M iodoacetamide. Unconjugated antibody was removed by size exclusion chromatography on an S300HR column equilibrated with 10 mM sodium borate +150 mM NaCl, pH 9.

Protein concentrations were determined from the absorbance at 280 and 405. The conjugate contained 0.5 mg/ml antibody and 0.3 mg/ml catalase.

### Example 15

Goat anti-mouse IgG-(HRP-dextran). 0.5 ml of goat anti-mouse IgG at 2 mg/ml (KPL #01-18-02) is labeled with a 25× molar excess of GMBS, desalted on a G25 column (GE Healthcare), equilibrated with PBS+5 mM EDTA, pH 6.8 and is concentrated with an Amicon Ultra 4 30 kDa cutoff device.

HRP-dextran is prepared and labeled with a 25 x molar excess of SATA

(Molecular BioSciences #57898). After 1 hr, the solution is made 25 mM hydroxylamine and desalted using an Amicon Ultra 15 30 kDa cutoff device, equilibrated with PBS +5 mM EDTA, pH 6.8.

The labeled HRP-dextran and antibody are combined at 1:1 HRP:antibody (wgt:wgt) and reacted for 1 hr. The solution is made 10 mM iodoacetamide and the pH raised to 9. Free antibody is removed using size exclusion chromatography on a S400HR column (GE Healthcare) (e.g., see FIG. 7).

### Example 16

Streptavidin polymer labeled with biotin-HRP and biotin-Mab. A 1 ml solution of VHMW dextran at 8 mg/ml in water is prepared. 80 ul of CDAP (100 mg/ml in acetonitrile) is added at time zero. 30 seconds later 0.25 M NaOH is added to maintain the pH at 9. At 3 minutes, 0.5 ml of a 20 mg/ml solution of streptavidin in 0.1 M sodium borate, pH 9 is added. After 2 hr, the solution is quenched by the addition of 150 ul of 2 M glycine, pH 9 and incubated for an additional 2 hr. Free streptavidin is removed by size exclusion chromatography on an S300HR column (GE Healthcare), equilibrated with PBS. The streptavidin concentration is determined using the BCA assay and the dextran concentration using the resorcinol/sulfuric acid assay.

A 10 mg/ml solution of HRP in 0.1 M HEPES, pH 8 is labeled with a 10× molar excess of sulfo-NHS-LC biotin (Pierce #21335). After 1 hr, the solution is dialyzed against PBS to remove free biotin. This product is biotin-HRP. A 10 mg/ml solution of a monoclonal antibody (Mab) in 0.1 M HEPES, pH 8 is labeled with a 10× molar excess of sulfo-NHS-LC biotin (Pierce #21335). After 1 hr, the solution is dialyzed against PBS to remove free biotin. This product is biotin-Mab.

Biotin-Mab and biotin-HRP are combined at equal molar ratio and then combined with streptavidin-dextran at a ratio of 1 mole of Mab per mole of streptavidin. After 15 min, the conjugate is purified on an S400HR size exclusion column (GE Healthcare). The product is (Mab+HRP)biotin-streptavidin/VHMWdextran (e.g., see FIG. 5).

### Example 17

Europium Fluorophore-dextran conjugates. VHMWdextran is prepared as above and functionalized with amino groups as described by Inman (J Immunol 114:704, 1975) to a level of approximately 500 umole amine per gram dextran. The europium fluorophore ABTA-Eu ([=Sodium [4′-(4′-Amino-4-biphenylyl)-2,2′:6′,2″-terpyridine-6,6″-diylbis(methyliminodiacetato)]europate(III)](TCI Chemical #A2083) is activated with cyanuric acid to form DBTA-EU as described in the product associated TCI Chemical literature: Dissolve 2 mg of ATBTA-Eu3+in 60 μl of 0.1M acetate buffer (pH 4.9). This solution is added 0.43 mg of cyanuric chloride in 25 μl of acetone, and stirred for 30 min. The reaction mixture is added drop wise to 1 ml of acetone, and formed precipitate is centrifuged. After washing with 0.5 ml of acetone twice, the yellow powder is dried in vacuum for 1 h. Dissolve the powder in lml of carbonate buffer gives (pH 9) for labeling. This solution contains ca. 2 mM of labeling reagent. The DBTA-Eu and amino-dextran are combined and reacted so that approximately 20 umole of amines/g dextran remain unlabeled. The DBTA-EU/amino-dextran is desalted to remove reagent. DBTA-EU/amino-dextran is made 50 mM HEPES, pH 7.3 and reacted with a 2 fold molar excess of GMBS over free amine. After 1 hr, the pH is reduced to 6.8 and desalted by dialysis against PBS, pH 6.8. The concentration is adjusted to 5 mg/ml dextran.

A monoclonal antibody (Mab) is prepared at 10 mg/ml in 50 mM HEPES, pH 8 and labeled with SPDP at 25× molar excess. After 1 hr, the pH is reduced to pH 6.8 and made 25 mM DTT. After 30 min, the solution is desalted on a G25 column, equilibrated with PBS +5 mM EDTA, pH 6.8. The void volume is concentrated to 10 mg/ml using an Amicon Ultra4, 10 kDa cutoff device.

The thiolated antibody and maleimide labeled europium fluorophore-dextran are combined at a 1:1 (wgt:wgt) ratio. After 2 hr, the solution is made 10 mM iodoacetamide and the pH raised to 9. After a 30 mM incubation, free antibody is removed by size exclusion chromatography on an S400HR column (GE Healthcare). (e.g., see FIGS. 4, 8 and 9).

Alternatively, streptavidin is thiolated with SPDP as above and combined with the maleimide derivatized europium fluorophore-dextran at a 1:1 (wgt:wgt) ratio. After 2 hr, the solution is made 10 mM iodoacetamide and the pH raised to 9. After a 30 min incubation, free antibody is removed by size exclusion chromatography on an S300HR column (GE Healthcare).

Other embodiments and uses of the invention will be apparent to those skilled in the art from consideration of the specification and practice of the invention disclosed herein. All references cited herein, including all publications, U.S. and foreign patents and patent applications and the priority provisional application, are specifically and entirely incorporated by reference. The term comprising, where ever used, is intended to include the terms consisting and consisting essentially of. It is intended that the specification and examples be considered exemplary only with the true scope and spirit of the invention indicated by the following claims.

1. A composition comprising a polymer with an average molecular weight of at least 50 kDa that is both water soluble and flexible to which is coupled to multiple detector or signal molecules at a ratio of detector or signal molecules to polymer of at least 10 to 1.

2. The composition of claim 1, wherein the polymer is high molecular weight form of polyacrylamide, dextran, Ficoll, pullunun, polyethylene glycol, a polyamino acid, or a combination thereof.

3. The composition of claim 1, wherein the average molecular weight is at least 100 kDa, at least 500 kDa, at least 1,000 kDa, at least 2,000 kDa, or at least 5,000 kDa.

4. The composition of claim 1, wherein the polymer is capable of bending at multiple points along a long axis of the polymer without breaking.

5. The composition of claim 1, wherein the multiple detector or signal molecules comprise greater than or equal to five, greater than or equal to ten, greater than or equal to twenty, greater than or equal to thirty, greater than or equal to fifty, greater than or equal to one hundred, greater than or equal to two and fifty hundred, or greater than or equal to five hundred.

6. The composition of claim 1, wherein the ratio is at least 50:1, at least 100:1, or at least 1,000:1.

7. The composition of claim 1, wherein the multiple detector or signal molecules are selected from the group consisting of antibodies or parts thereof, amino acids or peptides, avidin or streptavidin, luminescent molecules, receptor antigens, nucleic acid molecules, fluorescent molecules, radio-labeled molecules, enzyme-linked molecules, and magnetic molecules.

8. The composition of claim 1, wherein the polymer is coupled to the signal or detector molecules by covalent bonds, hydrogen bonds, van der Waals forces, a magnetic field, or a combination thereof.

9. The composition of claim 1, further comprising an adapter molecule coupled to the polymer wherein the multiple detector or signal molecules are coupled to the adaptor molecule.

10. The composition of claim 9, wherein the adaptor molecule is selected from the group consisting of biotin, oligonucleotides, peptides and oligopeptides.

11. The composition of claim 1, which comprises dextran as the polymer, and antibodies or parts thereof or straptavidin covalently linked to the polymer.

12. The composition of claim 11, which comprises biotin, enzyme-linked molecules or antibodies as the multiple detector or signal molecules.

13. A composition comprising a polymer with a molecular weight of at least 50 KDa that is both water soluble and flexible to which is coupled an adaptor molecule, wherein multiple detector or signal molecules are coupled to the adaptor molecule at a ratio detector or signal molecules to polymer of at least 10 to 1.

14. A method for the detection of an antigen comprising:
contacting a sample suspected of containing the antigen with a composition comprising a polymer with a molecular weight of at least 50 kDa that is both water soluble and flexible to which is coupled to multiple detector or signal molecules at a ratio of detector or signal molecules to polymer of at least 10 to 1;
incubating the sample and the composition for a period of time to form antigen-detector or signal molecule complexes;
detecting the presence of complexes formed after incubation; and
determining whether the antigen is present in the sample from detecting the presence of the complexes.

15. The method of claim 14, wherein the sample is a biological sample.

16. The method of claim 14, wherein the antigen is selected from the group consisting of biological molecules, peptides, proteins, receptors, and indicator molecules.

17. The method of claim 14, wherein the detection is quantitative.

18. The method of claim 14, wherein contacting comprises mixing the sample with the polymer.

19. The method of claim 14, wherein contacting comprises mixing equal parts by weight of sample with polymer.

20. The method of claim 14, wherein the incubation is for at least 5 seconds at room temperature or above.

21. The method of claim 14, wherein the incubation is for at least 5 minutes at above room temperature.

22. The method of claim 14, wherein the polymer is removed prior to detecting the presence of complexes.

23. The method of claim 14, wherein avidity of detection is increased at least two fold as compared to detection in an ELISA.

24. The method of claim 14, wherein avidity of detection is increased at least ten fold as compared to-detection in an ELISA.
